(19) United States
(12) Patent Application Publication
(10) Pub. No.: US 2021/0353747 A1
(43) Pub. Date: Nov. 18, 2021

(54) USE OF PLANT EXOSOMES FOR SHOWING MODULATING EFFECTS ON IMMUNE SYSTEM CELLS

(71) Applicant: YEDITEPE UNIVERSITESI, Istanbul (TR)

(72) Inventors: Fikrettin SAHIN, Istanbul (TR); Pakize Neslihan TASLI, Istanbul (TR); Batuhan Turhan BOZKURT, Istanbul (TR); Oguz Kaan KIRBAS, Istanbul (TR)

(73) Assignee: YEDITEPE UNIVERSITESI, Istanbul (TR)

(21) Appl. No.: 17/273,994

(22) PCT Filed: Sep. 3, 2019
(86) PCT No.: PCT/TR2019/050723

§ 371 Date: Mar. 5, 2021

(30) Foreign Application Priority Data
Sep. 6, 2018 (TR) 2018/12773

(51) Int. Cl. A61K 39/39; A61K 47/46

(52) U.S. Cl. CPC A61K 39/39; A61K 2039/55588; A61K 47/46

(57) ABSTRACT

Use of the effects of plant exosomes on the immune system as immune system enhancers, silencers and modulators against diseases is disclosed. The plant derived exosomes are obtained from at least one portion of the plant selected from the group consisting of the entire plant, fruit, leaf, seed, root, or differentiated tissues like the plant's tissue culture medium, stem cell, waste material, shell or phloem. In the scope of the invention, the plant exosomes having immunomodulatory effects are used mainly in autoimmune diseases, and in cell, tissue, organ transplantations and in Graft Versus Host disease as immune system enhancers, suppressors or, if necessary, as modulators performing both of the first two functions.

### CROSS REFERENCE TO THE RELATED APPLICATIONS

This application is the national phase entry of International Application No. PCT/TR2019/050723, filed on Sep. 3, 2019, which is based upon and claims priority to Turkish Patent Application No. 2018/12773, filed on Sep. 6, 2018, the entire contents of which are incorporated herein by reference.

### TECHNICAL FIELD

The present invention relates to use of the effects of plant exosomes on the immune system as immune system enhancers, silencers and modulators against diseases.

### BACKGROUND

The vesicles are small sacs which are involved in the transport and storage of substances within the cell and are separated by at least one lipid bilayer from the cytoplasm fluid. Exosomes are vesicles, which are released by many organisms from prokaryotes to high eukaryotes and plants, and which contain lipid bilayer vesicles of different sizes [1]. The importance of these vesicles lies beneath the capacity of transferring information to the other cells in order to influence the cell function. Signal transfer via exosomes is carried out by means of biomolecules in many different categories consisting of proteins, lipids, nucleic acid and sugars [2].

Functional interactions of extracellular vesicles with cells were first found in 1982 upon determining experimentally that vesicles isolated from seminal plasma increase sperm motility [3]. From this point on, studies have been conducted in many different tissues until today on the developments related to the molecular mechanism of vesicles and bringing the issues left in the dark into light.

The immune system is the body's defense mechanism and protects the body against infections and diseases. The immune system protects humans from many microorganisms, such as viruses, bacteria, fungi, protozoa and parasites, which are defined as microbes, and their harmful effects. The primary task of the immune system is to prevent these organisms from entering the body, to destroy the ones that have entered and to prevent or delay their spreading. The main element of the immune system is white blood cells. The white blood cells, which are also called white corpuscles or leukocytes, are produced in the bone marrow, lymph nodes, spleen and thymus gland. White blood cells having a diameter of 20 microns constitute an important part of the immune system by protecting the body against infectious diseases and foreign substances. Leukocytes are divided into two groups. Multinucleated ones are defined as Granulocytes and single nucleated ones are defined as Lymphocytes and Monocytes. Each white blood cell has its own defense mechanism. Neutrophils destroy disease-causing cells, Monocytes destroy the cells remaining from the dead tissues, Eosinophils destroy toxic substances, allergy-causing cells and parasites, Lymphocytes form the body's full immune system and protect it. In the blood of a healthy adult human containing one million cells, there are 4×10³-11×10³ leukocytes, in other words, one drop of blood approximately includes 4,000 to 11,000 leukocytes.

T cells are a type of white blood cells that controls cellular abnormalities and infections and plays an important role in the protection of the immune system. The destructive effects of a small number of T cells in the body are very significant in diseases such as HIV/AIDS. There are several different T cells. In general, they can be divided into two different types. Firstly, “the CD8 surface marker positive killer T cells” hunt and destroy the infected or cancerous cells. Secondly, “the CD4 surface marker positive helper T cells” modulate the response of the immune system and play an important role in all parts of the immune system.

To clarify the functions of the T cells, in our body, they
Check the intracellular environment for foreign invaders,
Fulfill the command to directly kill the virus infected or bacteria infected cells,
Fight to destroy the cancerous cells,
Help to activate the structure called antibody which is produced by the body to neutralize the pathogenic factors and
Remember the microbes encountered in the body many years ago and take precautions accordingly.

T cells are also responsible for rejection of the transplanted organ, immune system diseases such as diabetes and multiple sclerosis, and immune system responses such as allergic reactions, such as gluten intolerance. A low T cell number is more common than a high T cell number. Low T cell numbers usually indicate a problem in the immune system or lymph nodes. Low T-cell numbers can be seen in virus-induced infections such as influenza, and in case of immune system deficiency, radiation exposure, HIV and AIDS and in diseases affecting blood or lymph nodes such as leukemia. Chemotherapy drugs, radiation therapy, immunosuppressive drugs can affect the number of T cells.

B cells are lymphocytes that play a major role in the humoral immune response. The human body produces millions of different types of B cells every day, and each type has a specific receptor protein that can bind to a specific antigen in its membrane. CD19 is present as a marker in the entire B cell membrane. In the human body, millions of B cells circulate in the blood and lymph without producing antibodies. When any B cell encounters an antigen and receives additional signal from a helper T cell; it differentiates into one of the two different types of B cells described below. While B cells can be transformed into one of these cell types directly, they can also be transformed after an intermediate step.

Natural killer cells are non-specialized defense cells of the immune system. Natural killer cells make up about 10% of the lymphocytes in the blood. They lack the rearrangement of gene coding for antigen receptors found in T and B lymphocytes. Natural killer cells do not attack cells that show normal levels of MHC class I molecules, but kill foreign MHCs, such that they also kill those whose MHC I expression is reduced or absent. This is often seen in viral infections and cancer. Natural killer cells can be detected in peripheral blood as large lymphocytes containing red granules. The CD56 adhesion molecule is a marker of typical natural killer cells.

General purposes of use of adjuvants are as follows:
to enhance immunogenicity of antigens which are recombinant or obtained by thorough purification,
to produce a stronger and longer-lasting immune response in a short period of time,
to reduce the amount of antigens or the number of vaccinations required to achieve a primary immune response and thereby reducing the cost of vaccination,
to increase the activity of the vaccine in newborns, the elderly and people with immunodeficiency,
to reinforce the intake of antigens by the mucosa (stimulating the mucosal immunity),
to stimulate the cellular immunity,
to help prevent antigen competitiveness in combined vaccines.

A cellular immune response is required for protection against intracellular pathogenic microorganisms and a humoral immune response is required for protection against extracellular microorganisms. Aluminum salts are weak adjuvants, because they increase the antibody response by only stimulating the humoral immune response. They are sufficient for diseases that require a humoral immune response for protection (such as hepatitis B and whooping cough). However, they have no activity of stimulating cytotoxic T lymphocyte cells (cellular immunity). For example, in HIV vaccines the target is not to produce antibodies, but to stimulate cytotoxic lymphocytes. Furthermore, the aluminum compounds are not suitable for oral or intranasal administration. They cannot trigger the mucosal IgA response. On the contrary, they may cause allergic reactions in some people by increasing the IgE response. Therefore, many chemicals, biochemical substances and proteins produced as a result of immune system activity (such as cytokines) have been started to be investigated in the recent years as a potential adjuvant. However, local and systemic toxicity seen in most of these substances does not currently allow many of them to be used as adjuvants in human vaccines. The higher the adjuvant activity, the higher the incidence of side effects. Local side effects such as pain, inflammation, swelling, necrosis at the injection site, granulomas, sterile abscesses and lymphadenopathies, and systemic side effects such as nausea, fever, arthritis, uveitis, eosinophilia, allergy, anaphylaxis, immunosuppression and autoimmune diseases due to adjuvants may occur be encountered. In adjuvant studies, minimizing toxicity is seen as the most challenging step. For this reason, only aluminum adjuvants have continued to be used as adjuvants for human vaccines for nearly a century.

Regardless of what type of protection is desired, either cellular or humoral, an ideal adjuvant should provide the desired immunity; should provide immune memory, i.e. long-term immunity; should be safe and have minimum side effects; should not have any effect of stimulating autoimmunity; should not be mutagenic, carcinogenic, teratogenic; should be biodegradable; and should be inexpensive and have long shelf life.

Saponins are steroid or triterpenoid glycosides found in plants, some primitive marine organisms and bacteria. They are abundant in nature. Triterpenoid saponins are seen in soybeans, beans, peas, tea, spinach, sugar beet, licorice, sunflower, horse chestnut and ginseng, while steroid saponins are seen in oats, bell peppers, eggplants, tomato seeds, onions, asparagus, sweet potatoes and ginseng. Triterpenoid saponins comprise a hydrophobic nucleus and carbohydrate chains linked thereto. Saponin-containing adjuvants stimulate both cellular and humoral immunity. Low dosage is sufficient for adjuvant activity. Saponins also increase CD8 (+) cytotoxic lymphocyte response and strengthen the immune response to mucosal antigens. However, as they are surface active agents, they have been found to cause hemolysis in in vitro studies. There is a wide variety of adjuvants containing saponin.

Use of immunomodulatory chemicals is of great importance in carrying out and maintaining continuity of the cell or organ transplantations which are frequently performed today as a very important treatment method. Graft-versus-host disease (GVHD), which occurs especially in solid organ transplantation and hematopoietic stem cell transplantation, and which is characterized by severe immunological reaction mediated by healthy T-lymphocytes, is one of the diseases wherein immunomodulators are widely used. The most important reason for this is that it is the most important mortality and morbidity factor in the post-transplantation process. This disease, which can occur within the first three months after transplantation, has a significant effect mainly on the skin, liver and intestines. Although significant progress have been made from past to present in prevention thereof by means of immunosuppressive drugs such as cyclosporine and methotrexate; progress in treatment thereof has unfortunately been limited. In general, it is divided into two, namely acute and chronic GVHD, according to the time of onset after hematopoietic stem cell transplantation. Accordingly, it is important to confirm the diagnosis by biopsy since the treatment of chronic GVHD occurring after the 3^{rd} month involves high doses of immunosuppressive drugs and persists for a long time. In addition, infections that occur during the treatment process are one of the most common causes of death in patients with chronic GVHD. Therefore, administration of prophylactic antimicrobial drugs to these patients during immunosuppressive therapy has become a vital requirement. Studies on the development of different prophylactic and therapeutic methods are ongoing in the recent years in accordance with the findings of the pathogenesis of both acute and chronic GVHD.

Excessive or inappropriate reaction of the immune system when it encounters an antigen is called hypersensitivity. While the immune systems of healthy individuals can fight pathogens without causing much damage to their cells, the immune response of hypersensitive individuals causes damage to healthy tissues. Allergy, that is to say, the immune system's response to a harmless antigen falls into the scope of hypersensitivity. Eczema, allergic asthma and hay fever are among the most common chronic allergies.

In the treatment of hypersensitivity diseases, broad-spectrum anti-inflammatory drugs, anti-cytokine therapies and, particularly in more serious cases, treatments that reduce the number of lymphocyte cells in the body are used. Desensitization therapy may be used to reduce the sensitivity of individuals to the antigen to which they are allergic. Desensitization is achieved by administering the antigen, to which the individual is allergic, at controlled doses during the period of treatment, thereby building tolerance to the antigen.

The problems determined in the state of the art applications can be listed as follows:
An important problem for aluminum adjuvants is that their use is limited to bacterial and viral vaccines which require production of neutralizing antibodies,
Aluminum adjuvants may cause tendency to allergy and potential neurotoxicity due to increased IgE production,
Aluminum intoxication is also associated with amyotrophic lateral sclerosis and Alzheimer's disease,
Adjuvants containing plant-derived saponins lead to hemolysis in in vitro studies as they are surface active agents,
Immunosuppressive drugs such as cyclosporine and methotrexate make patients vulnerable to infectious diseases in long-term use,
Cyclosporine which is actively used in the treatment of GVHD causes significant nephrotoxicity and hypertension [4],
Subcutaneous injection of interferon beta (IFNB) administered in patients with multiple sclerosis (MS) is followed by first panniculitis and then local lipodystrophy at the injection site,
The drugs azathioprine and 6-mercaptopurine used for immunosuppressive purposes expose the body to hepatitis, pancreatitis and many other infections depending on the duration of use,
Various plant extracts containing phytochemicals such as flavonoids, polysaccharides, lactones, alkaloids, diterpenoids and glycosides have modulatory effects on the immune system; however, due to the high ratio of the chemical contaminants present in addition to the active ingredients, there are hesitations about their effective use and potential side effects.

South Korean patent application document no. KR20120002942, an application known in the state of the art, discloses microvesicles derived from the protoplast of cells and use thereof. These cells may be bacterial cells, archaea cells, mold cells, plant cells or L-form bacteria. A composition for delivery of a material for diagnosing or treating diseases or vaccine contains the micro vesicle. A method for manufacturing the micro vesicle comprises the steps of removing the cell walls from the cells to prepare protoplast, preparing micro vesicle in a suspension liquid containing the protoplast, and isolating micro vesicle from the suspension liquid.

The International patent application document no. WO2016166716 discloses an application known in the state of the art, discloses antineoplastic activity of nanovesicles isolated by Citrus limon. The process for obtaining nanovesicles from the Citrus plant comprises the steps of; a) centrifuging and filtering the juice in one or more consecutive cycles; b) ultracentrifuging the juice obtained in step (a) thus obtaining a supernatant and a sedimentation pellet containing the nanovesicles and recovering the vesicles. The process may further comprise the steps of c) subjecting the recovered pellet to a sucrose gradient ultracentrifugation; d) isolating the fraction having a density between 1.12 and 1.19 g/ml; e) optionally subjecting the fraction to ultracentrifugation; and f) optionally washing the pellet with physiological solution.

Chinese patent application document no. CN102697812 discloses an application known in the state of the art, discloses a method for extracting exosome from chicken bile and application of the said exosome in immunology. The exosome can be used as an adjuvant for immune reaction for immune-modulation.

The International patent application document no. WO2016168680, an application known in the state of the art, discloses a method for developing exosome based vaccines. The embodiments of the invention are directed to methods for making immunogenic compositions and methods of using such immunogenic compositions to treat various diseases.

Turkish patent application document no. TR 2012/05117 (EP1267924B1), an application in the state of the art, discloses immunotherapeutic methods and compositions. This invention relates to methods and compositions introducing chemical entities into antigen presenting cells. The resulting presentation of said antigens on the surface of the antigen presenting cells gives an effect on the immune system. The invention also relates to the resulting modified antigen presenting cells and pharmaceutical compositions containing these cells. The invention discloses new understandings in adjuvancy and adjuvant preparations, including a series of related peptides and phospholipid vesicles incorporating said peptides.

Turkish patent application document no. TR 2016/08391 (EP2591802B1), an application in the state of the art, discloses vaccine compositions based on sticholysin encapsulated in liposomes. The said invention relates to the field of Biotechnology applied to human health. Here it is described a vaccine vehicle wherein toxins from eukaryotic organisms are encapsulated into multilamellar vesicles obtained by the dehydration-rehydration procedure whose lipidic composition is dipalmitoylphosphatidylcholine:cholesterol in a 1:1 molar ratio for subcutaneous or intramuscular administration. These compositions do not require the use of other adjuvants. The disclosed compositions allow modulation of CTL-specific immune response against one or several antigens co-encapsulated into toxin-containing liposomes. The vaccinal vehicle of the said invention shows advantages over others disclosed by the previous art due to the robustness and functionality of the induced immune response as well as its immunomodulating properties.

The patent applications numbered U.S. Pat. No. 9,717,733, WO2006007529, WO1999003499, WO2017004526 and US20100092524 are also applications in the state of the art.

### SUMMARY

The objective of the present invention is to use plant exosomes isolated from plant lysates as immune system enhancers, silencers and modulators against diseases originating from immune system disorders.

Another objective of the present invention is to provide a higher suppression compared to that obtained by substances obtained from similar activators and chemicals currently used in suppressing the activation of blood cells.

A further objective of the present invention is to provide a higher activation compared to that obtained by substances obtained from similar activators and chemicals currently used for enhancing the activation of blood cells.

Another objective of the present invention is to provide an immunomodulatory agent which does not exert toxic effects to the liver and the other organs like the other drugs as it is completely of plant origin.

### BRIEF DESCRIPTION OF THE DRAWINGS

“Use of Plant Exosomes for Showing Modulatory Effects on Immune System Cells” developed to fulfill the objective of the present invention is illustrated in the accompanying figures wherein,

FIG. 1A shows the graphical representation of the characterization of the exosome isolated from the Warty-Leaved Rhubarb plant in the scope of the present invention (flow cytometry graph measured by a control group, by flow cytometry device); FIG. 1B shows the graphical representation of the characterization of the exosome isolated from the Warty-Leaved Rhubarb plant in the scope of the present invention (flow cytometry graph measured by CD9 antibody, which is an exosome characterization marker, by flow cytometry device); FIG. 1C shows the graphical representation of the characterization of the exosome isolated from the Warty-Leaved Rhubarb plant in the scope of the present invention (flow cytometry graph measured by HSP70 antibody, which is an exosome characterization marker, by flow cytometry device); FIG. 1D shows the graphical representation of the characterization of the exosome isolated from the Warty-Leaved Rhubarb plant in the scope of the present invention (flow cytometry graph measured by CD63 antibody, which is an exosome characterization marker, by flow cytometry device); FIG. 1E shows the graphical representation of the characterization of the exosome isolated from the Warty-Leaved Rhubarb plant in the scope of the present invention (exosome diameter measurement graph);

FIG. 2A shows the graphical representation of the characterization of the exosome isolated from Celery in the scope of the present invention (flow cytometry graph measured by a control group, by flow cytometry device); FIG. 2B shows the graphical representation of the characterization of the exosome isolated from Celery in the scope of the present invention (flow cytometry graph measured by CD9 antibody, which is an exosome characterization marker, by flow cytometry device); FIG. 2C shows the graphical representation of the characterization of the exosome isolated from Celery in the scope of the present invention (flow cytometry graph measured by HSP70 antibody, which is an exosome characterization marker, by low cytometry device); FIG. 2D shows the graphical representation of the characterization of the exosome isolated from Celery in the scope of the present invention (flow cytometry graph measured by CD63 antibody, which is an exosome characterization marker, by flow cytometry device); FIG. 2E shows the graphical representation of the characterization of the exosome isolated from Celery in the scope of the present invention (exosome diameter measurement graph);

FIG. 3A shows the graphical representation of the characterization of the exosome isolated from Pomegranate in the scope of the present invention (flow cytometry graph measured by a control group, by flow cytometry device);

FIG. 3B shows the graphical representation of the characterization of the exosome isolated from Pomegranate in the scope of the present invention (flow cytometry graph measured by CD9 antibody, which is an exosome characterization marker, by flow cytometry device); FIG. 3C shows the graphical representation of the characterization of the exosome isolated from Pomegranate in the scope of the present invention (flow cytometry graph measured by HSP70 antibody, which is an exosome characterization marker, by flow cytometry device); FIG. 3D shows the graphical representation of the characterization of the exosome isolated from Pomegranate in the scope of the present invention (flow cytometry graph measured by CD63 antibody, which is an exosome characterization marker, by flow cytometry device); FIG. 3E shows the graphical representation of the characterization of the exosome isolated from Pomegranate in the scope of the present invention (exosome diameter measurement graph);

FIG. 4A shows the graphical representation of the characterization of the exosome isolated from Leek in the scope of the present invention (flow cytometry graph measured by a control group, by flow cytometry device); FIG. 4B shows the graphical representation of the characterization of the exosome isolated from Leek in the scope of the present invention (flow cytometry graph measured by CD9 antibody, which is an exosome characterization marker, by flow cytometry device); FIG. 4C shows the graphical representation of the characterization of the exosome isolated from Leek in the scope of the present invention (flow cytometry graph measured by HSP70 antibody, which is an exosome characterization marker, by flow cytometry device); FIG. 4D shows the graphical representation of the characterization of the exosome isolated from Leek in the scope of the present invention (flow cytometry graph measured by CD63 antibody, which is an exosome characterization marker, by flow cytometry device); FIG. 4E shows the graphical representation of the characterization of the exosome isolated from Leek in the scope of the present invention (exosome diameter measurement graph);

FIG. 5A shows the graphical representation of the characterization of the exosome isolated from Horseradish in the scope of the present invention (flow cytometry graph measured by a control group, by flow cytometry device); FIG. 5B shows the graphical representation of the characterization of the exosome isolated from Horseradish in the scope of the present invention (flow cytometry graph measured by CD9 antibody, which is an exosome characterization marker, by flow cytometry device); FIG. 5C shows the graphical representation of the characterization of the exosome isolated from Horseradish in the scope of the present invention (flow cytometry graph measured by HSP70 antibody, which is an exosome characterization marker, by flow cytometry device); FIG. 5D shows the graphical representation of the characterization of the exosome isolated from Horseradish in the scope of the present invention (flow cytometry graph measured by CD63 antibody, which is an exosome characterization marker, by flow cytometry device); FIG. 5E shows the graphical representation of the characterization of the exosome isolated from Horseradish in the scope of the present invention (exosome diameter measurement graph);

FIG. 6A shows the graphical representation of the characterization of the exosome isolated from Ginger in the scope of the present invention (flow cytometry graph measured by a control group, by flow cytometry device); FIG. 6B shows the graphical representation of the characterization of the exosome isolated from Ginger in the scope of the present invention (flow cytometry graph measured by CD9 antibody, which is an exosome characterization marker, by flow cytometry device); FIG. 6C shows the graphical representation of the characterization of the exosome isolated from Ginger in the scope of the present invention (flow cytometry graph measured by HSP70 antibody, which is an exosome characterization marker, by flow cytometry device); FIG. 6D shows the graphical representation of the characterization of the exosome isolated from Ginger in the scope of the present invention (flow cytometry graph measured by CD63 antibody, which is an exosome characterization marker, by flow cytometry device); FIG. 6E shows the graphical representation of the characterization of the exosome isolated from Ginger in the scope of the present invention (exosome diameter measurement graph);

FIG. 7 shows the graphical representations of the effect of the Warty-Leaved Rhubarb exosome on white blood cells in the scope of the present invention (the graph of measurement of CD4 T helper lymphocytes. CD8 T cytotoxic lymphocytes, CD19 B lymphocytes and CD56 natural killer cells with the antibodies that are surface markers by flow cytometry device);

FIG. 8 shows the graphical representations of the effect of the Warty-Leaved Rhubarb exosome on IL2 activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 9 shows the graphical representations of the effect of the Warty-Leaved Rhubarb exosome on PHA activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 10 shows the graphical representations of the effect of the Warty-Leaved Rhubarb exosome on Mite Allergen activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 11 shows the graphical representations of the effect of the Warty-Leaved Rhubarb exosome on Pollen Allergen activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 12 shows the graphical representation of the effect of the Celery exosome on white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 13 shows the graphical representations of the effect of the Celery exosome on IL2 activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 14 shows the graphical representations of the effect of the Celery exosome on PHA activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 15 shows the graphical representations of the effect of the Celery exosome on Mite Allergen activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 16 shows the graphical representations of the effect of the Celery exosome on Pollen Allergen activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 17 shows the graphical representation of the effect of the Pomegranate exosome on white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 18 shows the graphical representations of the effect of the Pomegranate exosome on IL2 activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 19 shows the graphical representations of the effect of the Pomegranate exosome on PHA activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 20 shows the graphical representations of the effect of the Pomegranate exosome on Mite Allergen activated white blood cells in the scope of the present invention (the graph of measurement of CD4 T helper lymphocytes. CD8 T cytotoxic lymphocytes, CD19 B lymphocytes and CD56 natural killer cells with the antibodies that are surface markers by flow cytometry device);

FIG. 21 shows the graphical representations of the effect of the Pomegranate exosome on Pollen Allergen activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 22 shows the graphical representation of the effect of the Leek exosome on white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 23 shows the graphical representations of the effect of the Leek exosome on IL2 activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 24 shows the graphical representations of the effect of the Leek exosome on PHA activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 25 shows the graphical representations of the effect of the Leek exosome on Mite Allergen activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 26 shows the graphical representations of the effect of the Leek exosome on Pollen Allergen activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 27 shows the graphical representation of the effect of the Horseradish exosome on white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 28 shows the graphical representations of the effect of the Horseradish exosome on IL2 activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 29 shows the graphical representations of the effect of the Horseradish exosome on PHA activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 30 shows the graphical representations of the effect of the Horseradish exosome on Mite Allergen activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 31 shows the graphical representations of the effect of the Horseradish exosome on Pollen Allergen activated white blood cells in the scope of the present invention (the graph of measurement of CD4 T helper lymphocytes. CD8 T cytotoxic lymphocytes, CD19 B lymphocytes and CD56 natural killer cells with the antibodies that are surface markers by flow cytometry device);

FIG. 32 shows the graphical representation of the effect of the Ginger exosome on white blood cells in the scope of the present invention (the graph of measurement of CD4 T helper lymphocytes, CD8 T cytotoxic lymphocytes. CD19 B lymphocytes and CD56 natural killer cells with the antibodies that are surface markers by flow cytometry device);

FIG. 33 shows the graphical representations of the effect of the Ginger exosome on IL2 activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 34 shows the graphical representations of the effect of the Ginger exosome on PHA activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device);

FIG. 35 shows the graphical representations of the effect of the Ginger exosome on Mite Allergen activated white blood cells in the scope of the present invention (the graph of measurement of CD4 T helper lymphocytes, CD8 T cytotoxic lymphocytes, CD19 B lymphocytes and CD56 natural killer cells with the antibodies that are surface markers by flow cytometry device); and

FIG. 36 shows the graphical representations of the effect of the Ginger exosome on Pollen Allergen activated white blood cells in the scope of the present invention (the graph of measurement of CD4 T helper lymphocytes. CD8 T cytotoxic lymphocytes, CD19 B lymphocytes and CD56 natural killer cells with the antibodies that are surface markers by flow cytometry device).

The subject matter of the present invention is to use plant exosomes isolated from plant lysates as immune system enhancers, silencers and modulators against diseases affecting immune system. The plant exosomes of the present invention are plant derived exosomes that enable prevention and treatment of diseases by activating, suppressing and modulating the immune system cells.

The effects of the plant exosomes of the present invention on the immune system are based on the effect on the division of immune system cells. The plant exosomes that activate the immune system stimulate the immune system cells enabling them to proliferate, thereby strengthening the immune response. The suppressive plant exosomes, on the other hand, make the immune system cells insensitive to division, thereby suppressing the immune system response. These effects of the exosomes discussed in the invention are shown in the data presented by changes in the surface proteins of the immune system cells to which they are applied (CD 8, CD 19 and CD 56).

In the scope of the invention, plant exosomes are used mainly in autoimmune diseases, and in cell, tissue, organ transplantations and in Graft Versus Host disease as immune system enhancers, suppressors or, if necessary, as modulators performing both of the first two functions.

Within the scope of the invention, the plant exosomes are obtained from at least one portion of the plant selected from the group consisting of the entire plant, fruit, leaf, seed, root or differentiated tissues like culture medium of the plant, stem cell, waste material, shell or phloem. Plant tissue culture is preferred as a source from which plant exosomes are obtained in order to produce exosomes at a concentration up to 5 times higher than the exosomes obtained from similar plants and to maintain the content and properties of the produced exosomes for a long time, thereby preventing them from being affected by the effects of the farm, harvesting, transport, etc.

The plant exosomes of the present invention are transfected to enable the cells in the plant tissue culture to produce proteins enhancing, suppressing or modulating the immunity system. Transfection is an external gene transfer to a cell. Transfection of the cells in plant culture can enable them to produce target proteins, and the exosomes secreted by these cells can thus contain these proteins.

Within the scope of the invention, the plant exosomes are isolated by an isolation method selected from the group consisting of isolation by two phase liquid system, graduated centrifuge, ultrafiltration, chromatographic methods, polymer based isolation and isolation by microbeads. Among them, the purest exosome isolation is achieved by isolation with two phase liquid system and therefore this isolation method is preferred within the scope of the present application.

The plant-derived exosomes of the present invention are used mainly in autoimmune diseases, and in cell, tissue, organ transplantations, in Graft Versus Host disease, and diseases where the immune system is affected such as rheumatoid arthritis, juvenile rheumatoid arthritis, systemic lupus erythematosus, sclerosis, Sjogren's syndrome, Type 1 Diabetes, allergic asthma, Wegener granulomatosis, Multiple Sclerosis, Crohn's disease, psoriasis, Graves' disease, Celiac Disease, alopecia areata (pelade), central nervous system vasculitis, Hashimoto's thyroiditis, myasthenia gravis, Goodpasture's syndrome, autoimmune hemolytic anemia, Guillan-Barre syndrome, polyarteritis nodosa, idiopathic thrombocytic purpura, temporal arteritis, primary biliary cirrhosis, Addison's Disease, ankylosing spondylitis, Reiter's syndrome, Takayazu's arteritis, and vitiligo as immune system enhancers, suppressors or, if necessary, modulators performing both of the first two functions. Plant-derived exosomes are administered orally, intranasally, intravenously, intramuscularly, intradermally, topically, intraperitoneally, and by injection for administering the effective dose of the selected plant exosomes to the patient to treat immune system-mediated diseases. They are also used for purposes of carrying immunomodulatory drugs, as an adjuvant for vaccination applications, and as a nutritional supplement for the purpose of modulating the immune system.

Method of isolation via two phase liquid system used for isolation of the plant exosomes used in the scope of the present invention comprises following steps:
Disintegrating the plant, whose exosome will be isolated, by a blender to obtain the lysate thereof,
Centrifuging at a stirring rate of 2,000 g to 10,000 g for 5-20 minutes for isolation of the exosomes from the plant lysate,
Removing the particles of size 220 nm and above by filtration after centrifugation,
Transferring the exosome-protein mixture obtained by centrifugation into a two phase liquid system containing PEG phase and DEX phase for separation thereof,
Removing the nonexosomal proteins, cellular fat and other impurities from the exosomes by utilizing the chemical tendency of the PEG phase to the proteins and the DEX phase to the phospholipid structured membranes,
Obtaining the isolated exosomes.

The present invention is for utilizing the effects of plant exosomes on the immune system as immune system enhancers, silencers and modulators against diseases. In the scope of the invention, the immunomodulatory effects of the plant exosomes are used mainly in autoimmune diseases, and in cell, tissue, organ transplantations and in diseases such as Graft Versus Host disease as immune system enhancers, suppressors or, if necessary, as modulators performing both of the first two functions. The effects of plant exosomes can vary according to the plant from which the exosome is isolated. While these can be the entire plant, fruit, leaf, seed and root, they may also be differentiated tissues like the plant's culture medium, stem cell, waste material, shell or phloem. The plant exosomes can be isolated by many methods such as isolation by two phase liquid system, graduated centrifuge, ultrafiltration, chromatographic methods, polymer based isolation and isolation by microbeads. The exosomes used in the study conducted within the scope of the invention are isolated from the plants disclosed in Table 1.

TABLE 1
The plants used for isolation of exosomes
within the scope of the invention.

| Turkish | English | Latin |
|---|---|---|
| Nar | Pomegranate | Punica granatum |
| Pirasa | Leek | Allium ampeloprasum |
| Kereviz | Celery | Apium graveolens |
| Kara Turp | Horseradish | Radix Raphani nigri |
| Eşgin | Warty-Leaved | Reun Ribes |
| | rhubarb | |
| Zencefil | Ginger | Zingiber officinale |

The large size particles resulting from plant disintegration by centrifugation performed between 2,000 g and 10,000 g for 5-20 minutes for exosome isolation from plant lysate are intended not to cause any impurities in the dextran phase upon precipitating due to the centrifugation applied during the two phase separation process and their weights. In addition, it is ensured that the filter, which is used during the filtration process carried out for removing particles sized 220 nanometers and above, is not clogged. Exosomes are cleared of nonexosomal proteins, cellular fats and other impurities by utilizing the chemical tendency of the PEG phase to the proteins and the DEX phase to the phospholipid structured membranes in the two-phase liquid system. The DEX phase formed by means of the concentrations of the polymers that are used in the solution separate the exosomes.

### DETAILED DESCRIPTION OF THE EMBODIMENTS

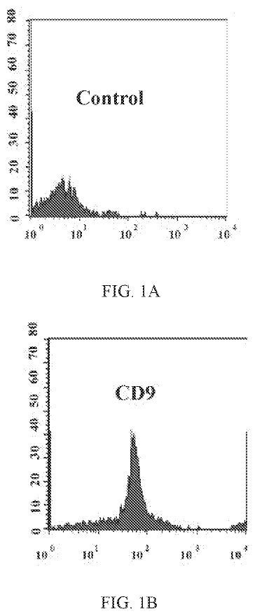

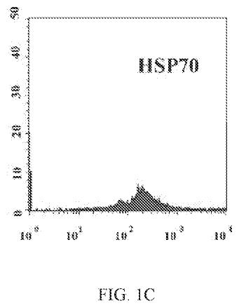

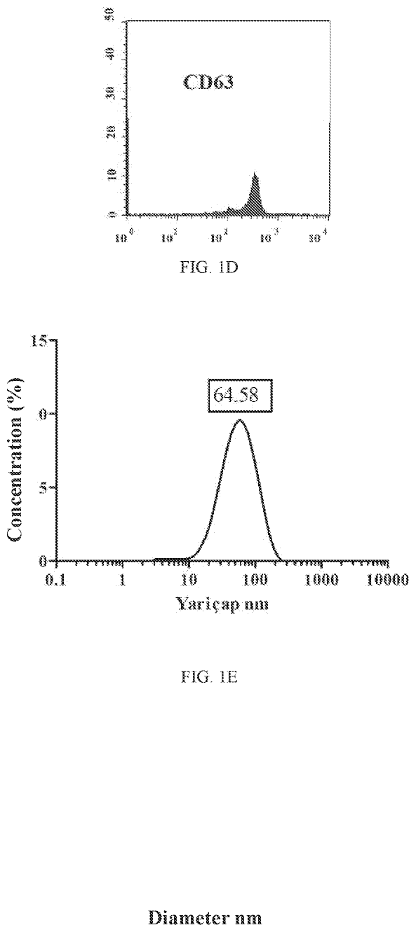

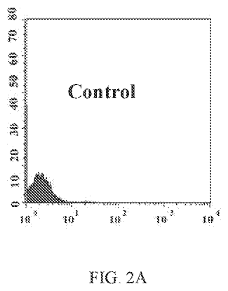

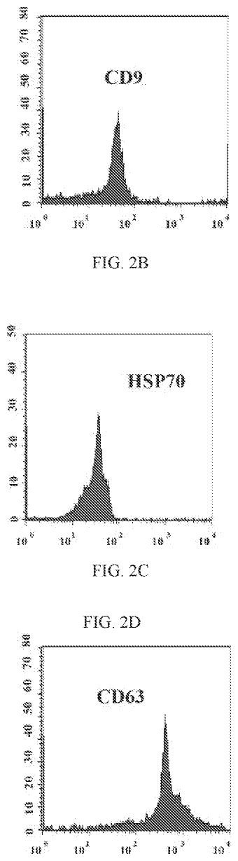

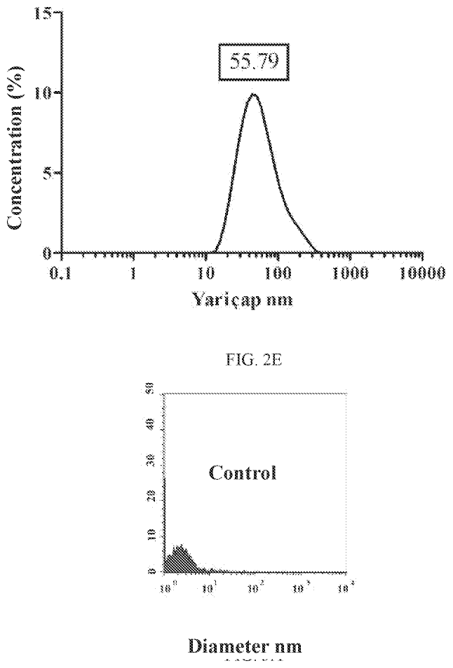

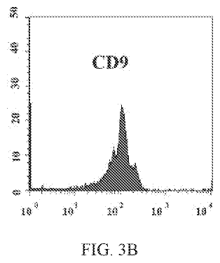

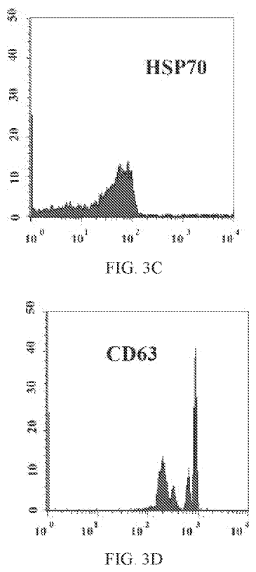

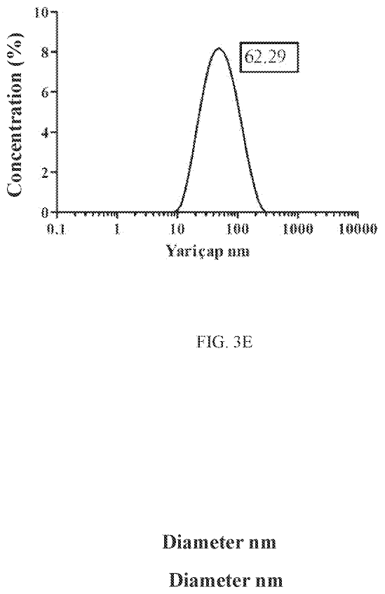

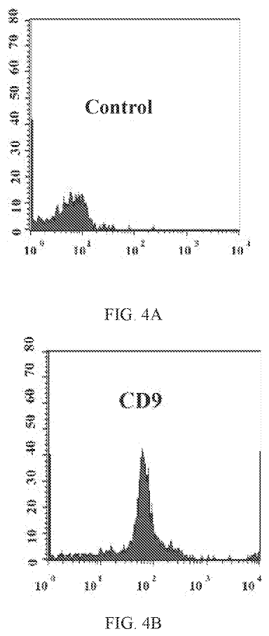

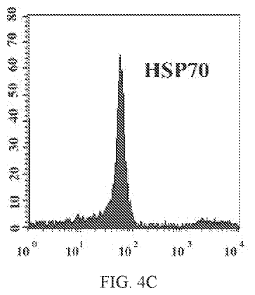

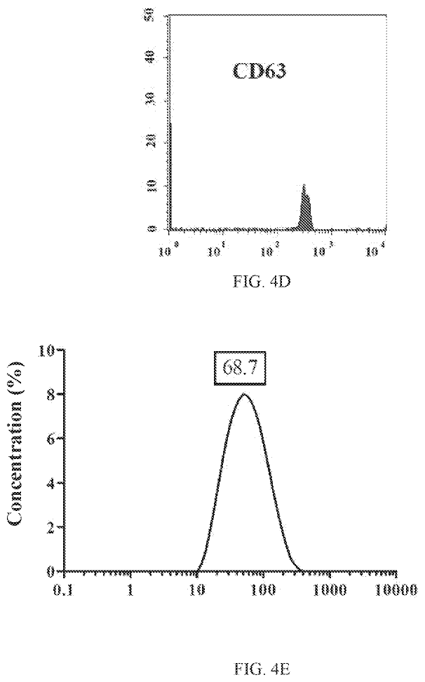

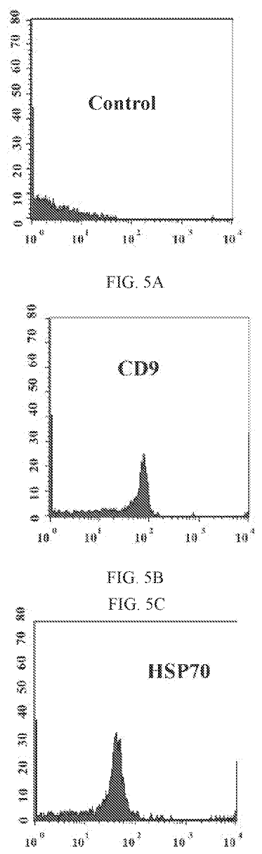

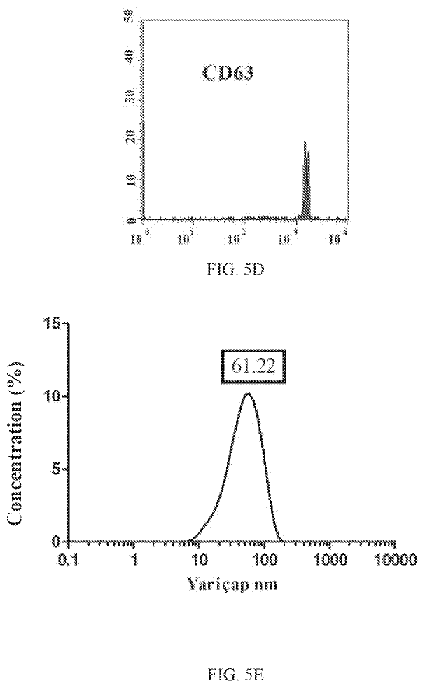

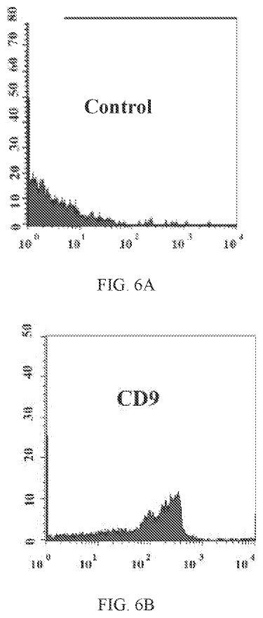

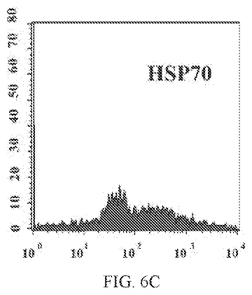

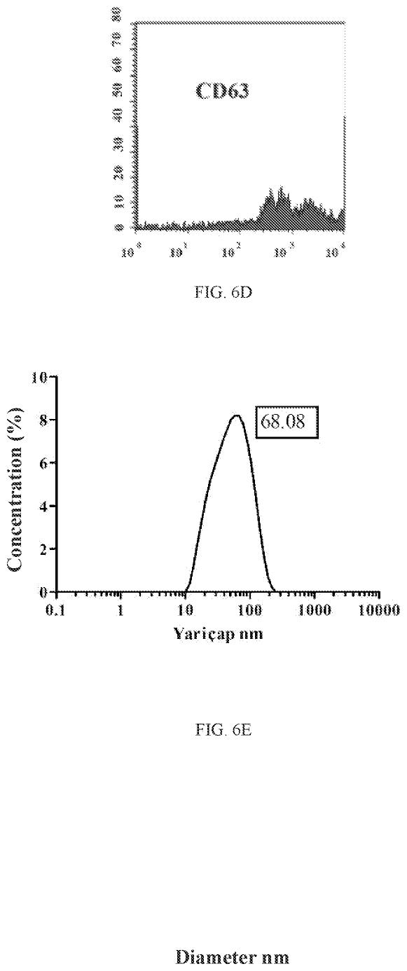

The isolated exosomes are marked by the surface markers CD9, CD63 and HSP70 antibodies which are carried by the exosomes and the exosomes carrying these markers are measured by flow cytometry device. At the same time, the dimensions of the exosomes isolated with the Zeta Sizer device are also measured FIGS. 1A-E to 6A-E.

White blood cell isolation is performed to determine the effects of the plant exosomes on the blood cells. Blood introduced into a tube containing EDTA or a solution enabling blood clotting is mixed with PBS at a ratio of 1:1 by volume. It is carefully poured onto the ficoll solution in another tube without mixing the phases. The tube containing blood, PBS and ficoll is centrifuged for 15 minutes at approximately 3000 RPM. After centrifugation, the white intermediate phase containing the white blood cells between the upper plasma and the ficoll is withdrawn and transferred to a clean tube and it is washed by adding approximately 10 mL of PBS thereon. The cells are centrifuged again at 1500 RPM for 5 minutes. The cell pellet is removed and the white blood cell is cultured in the medium. These isolated white blood cells include cells which play a role in the immune system such as T cells, B cells, natural killer cells, and dendritic cells.

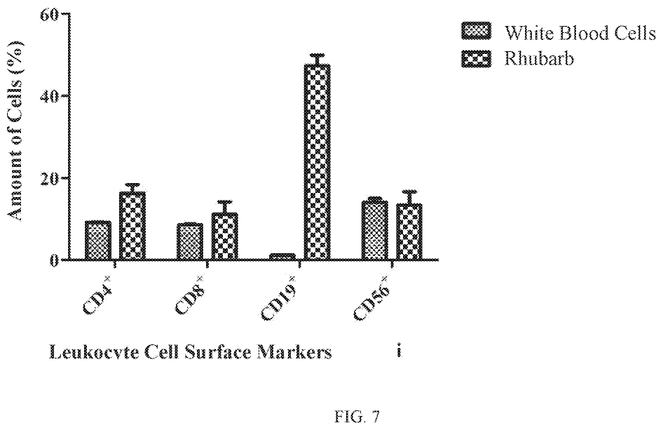

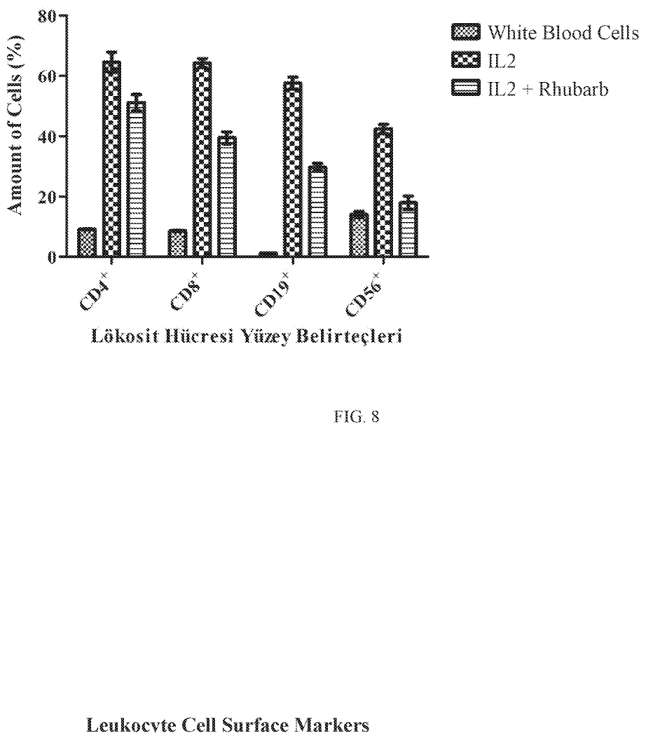

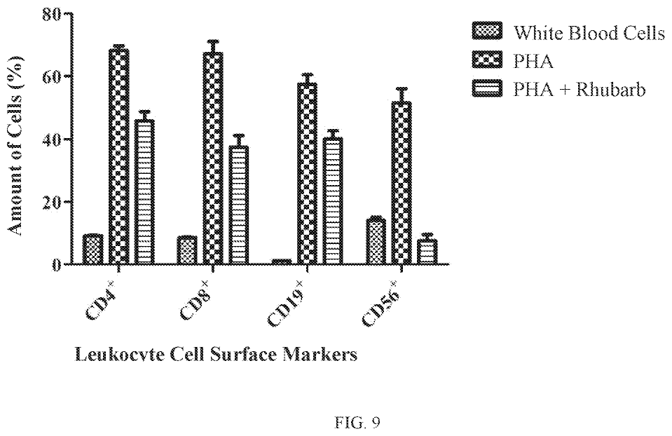

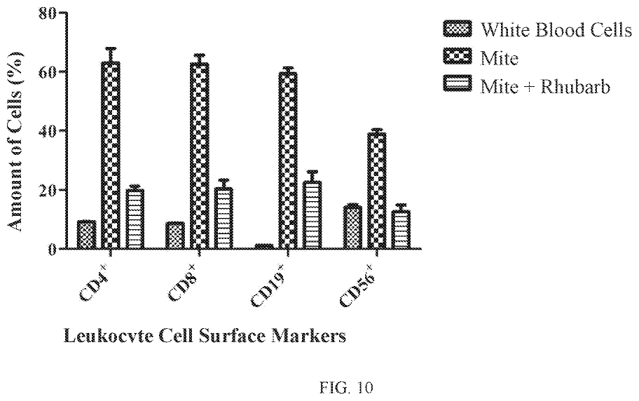

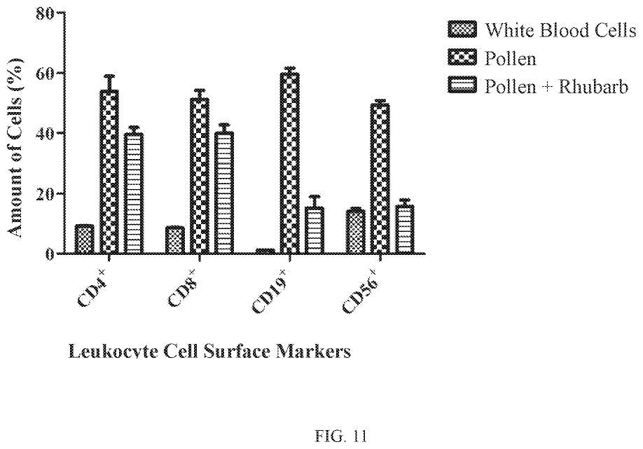

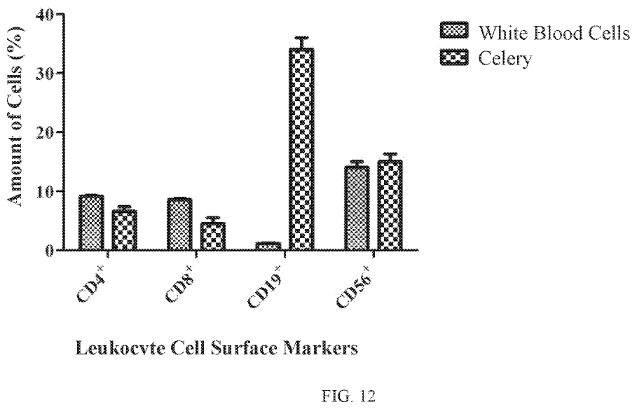

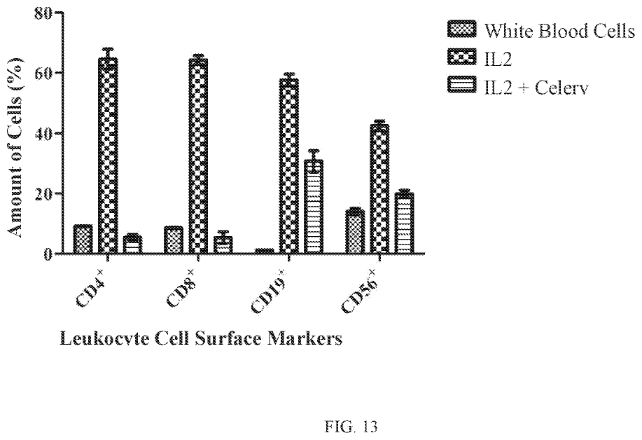

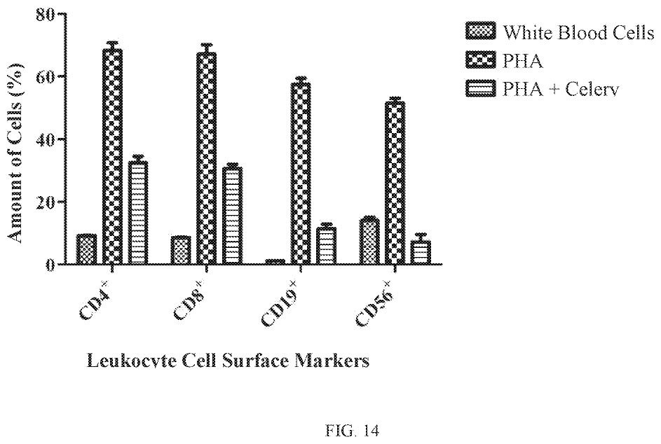

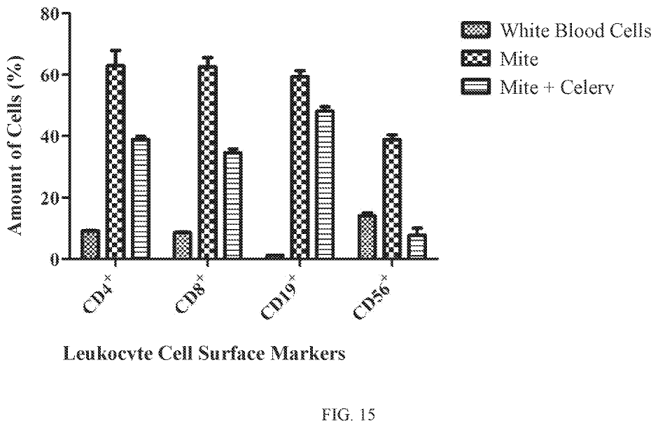

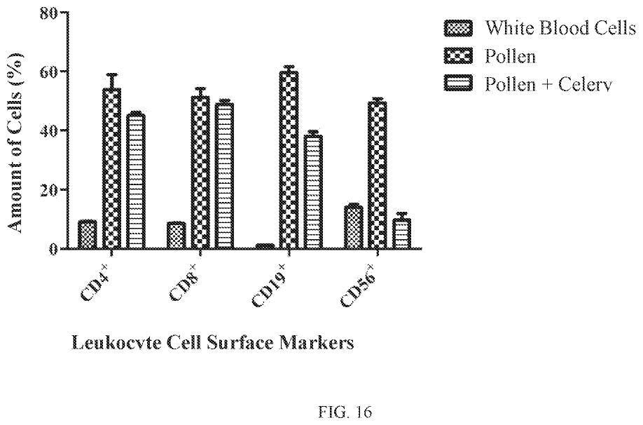

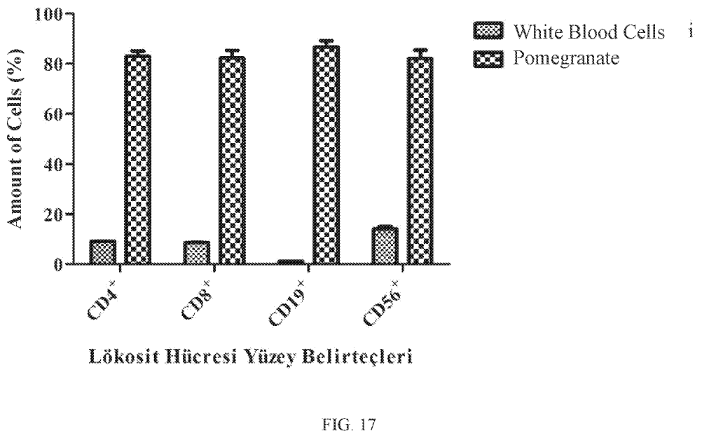

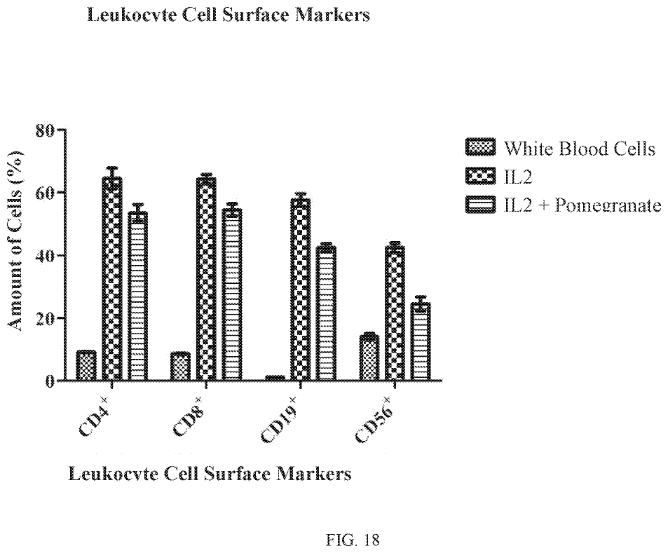

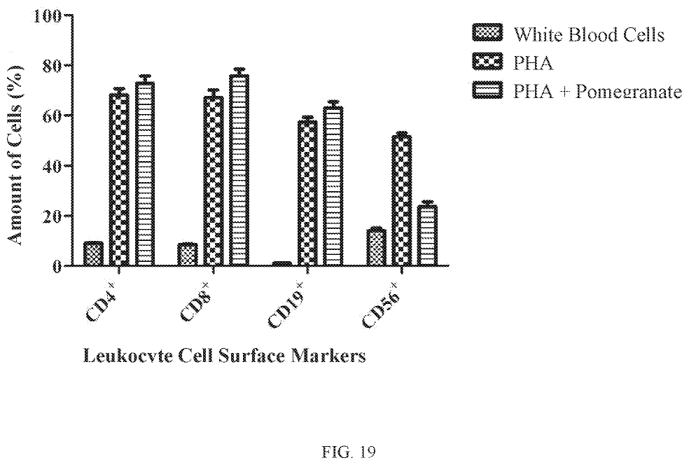

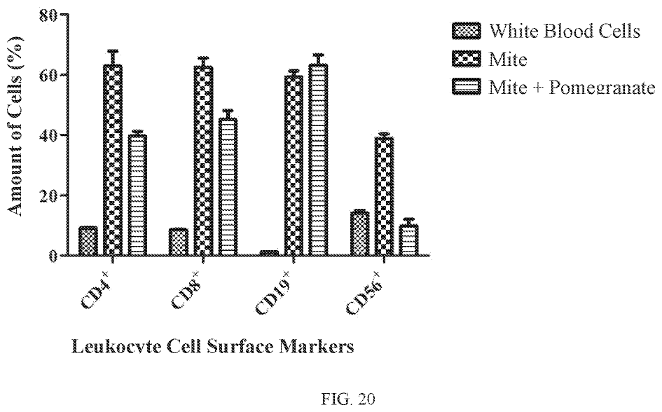

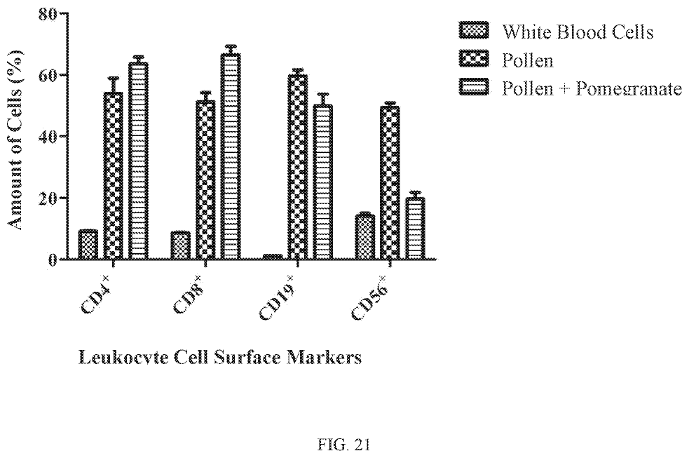

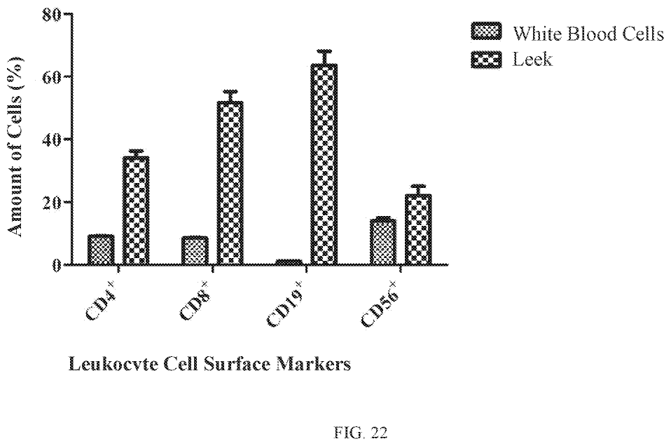

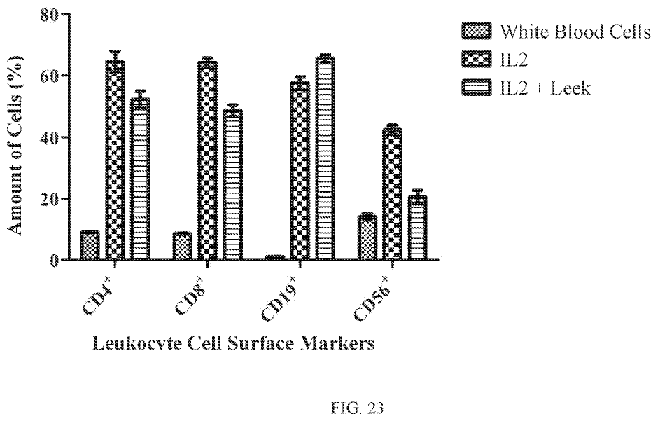

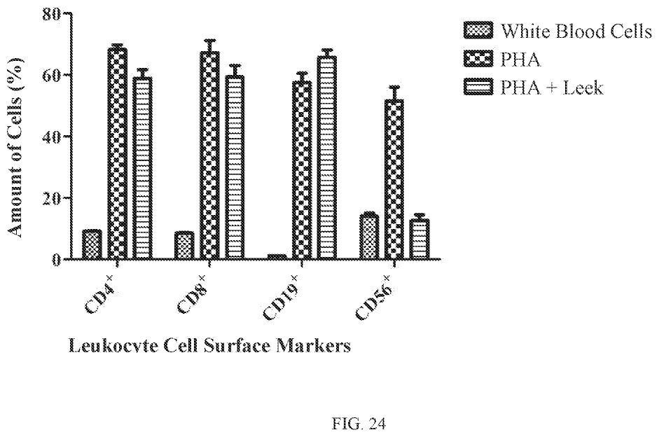

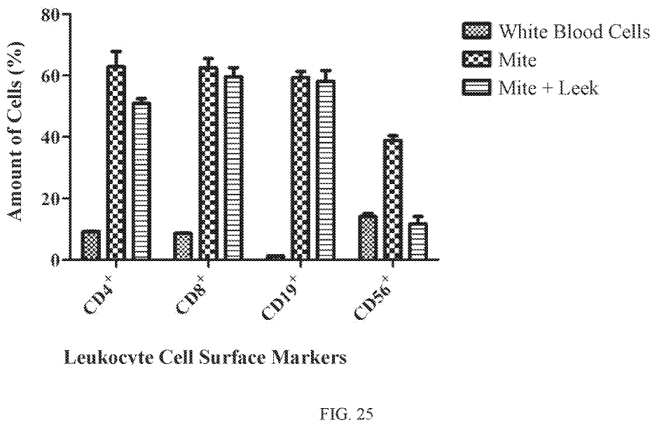

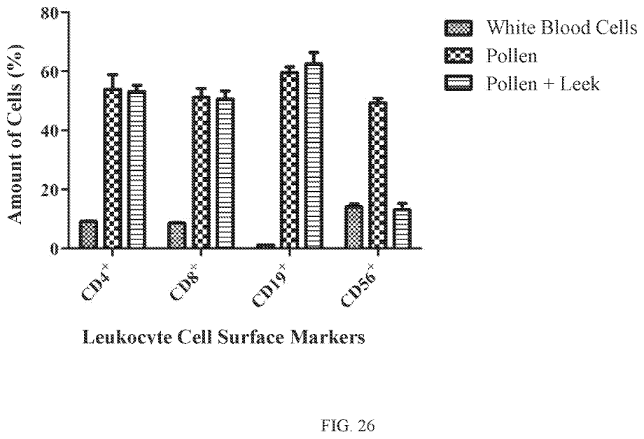

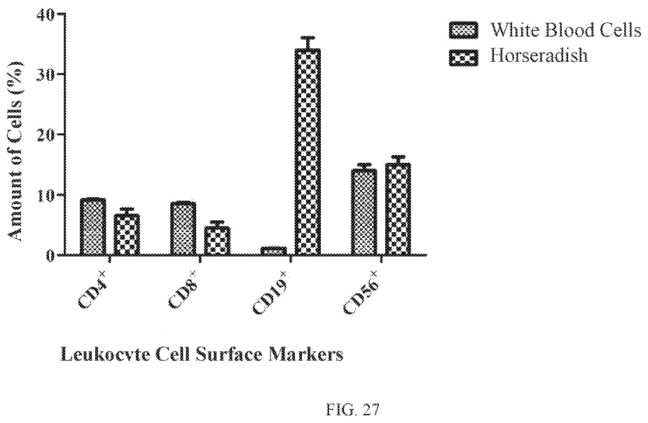

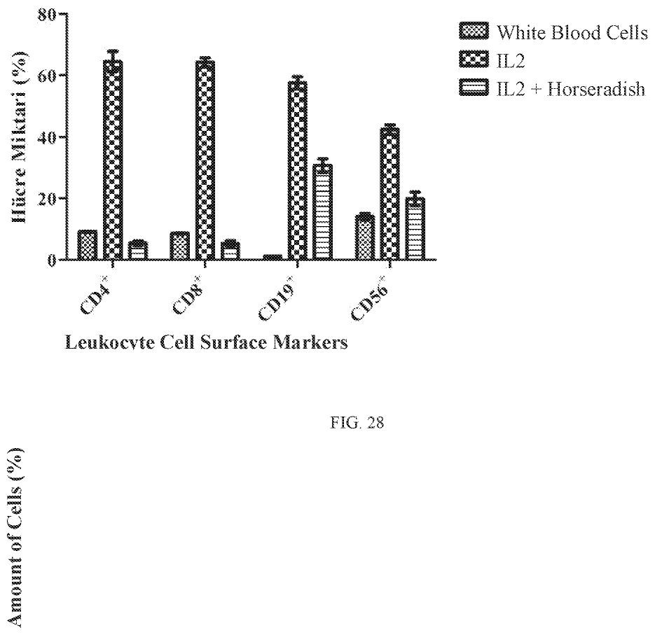

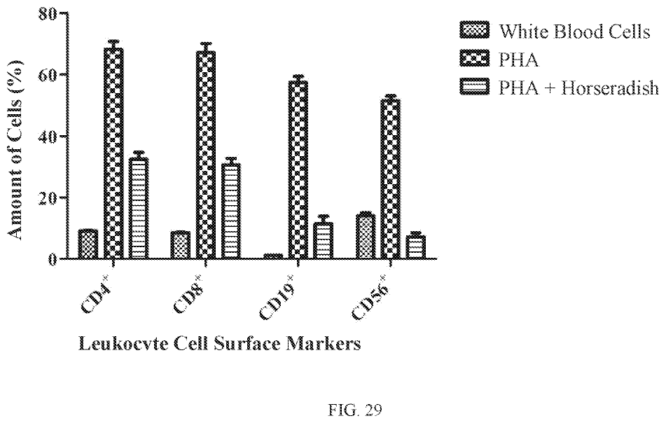

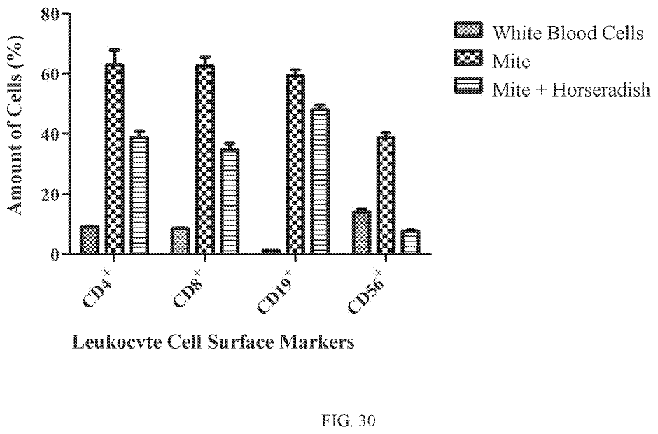

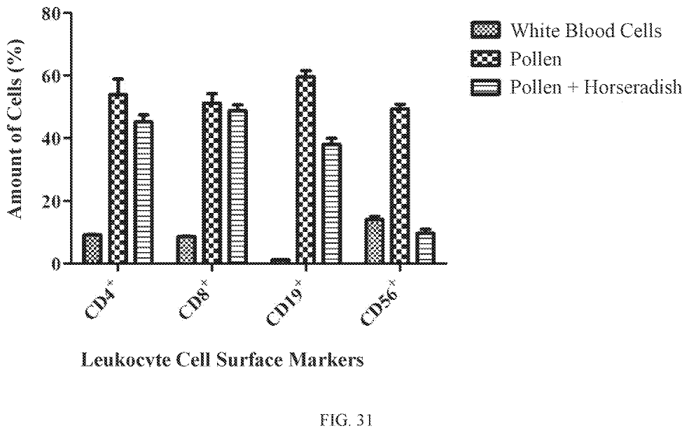

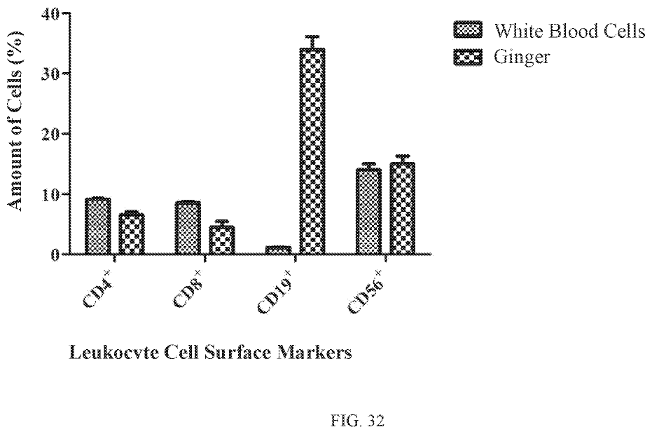

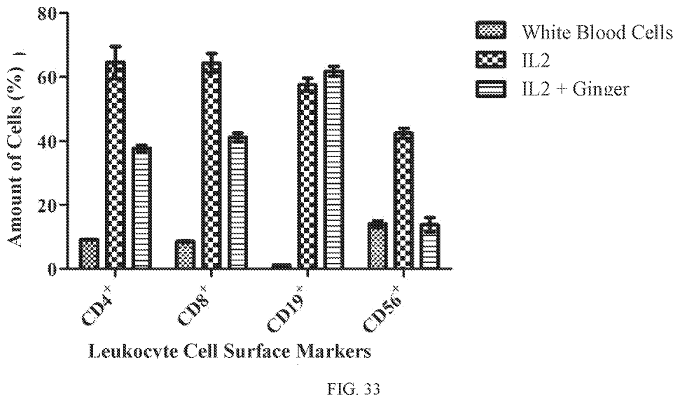

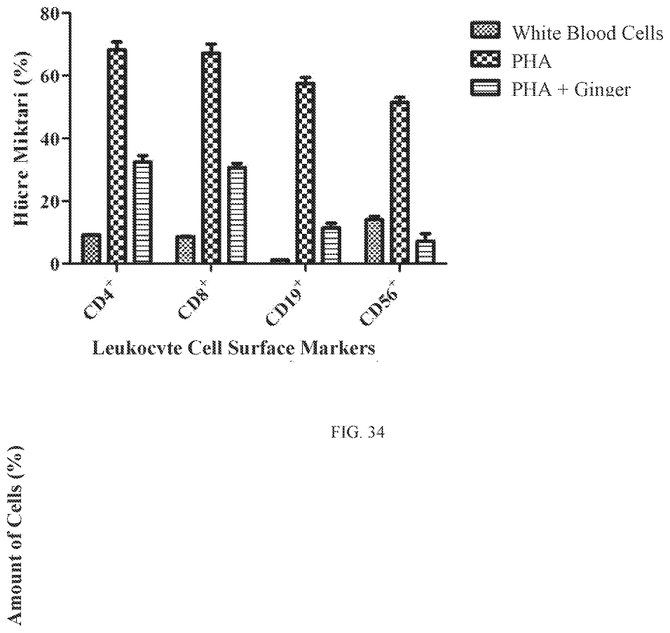

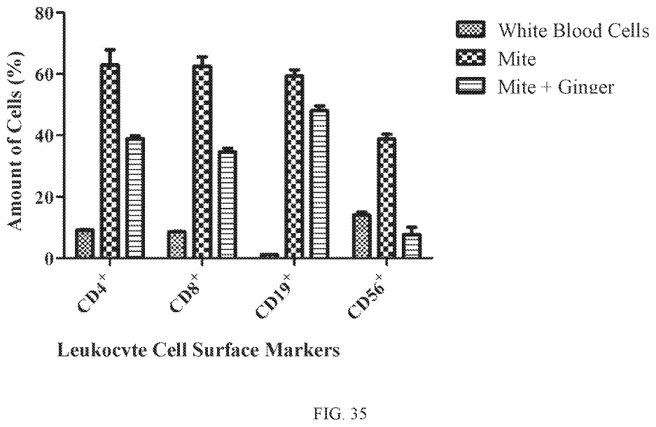

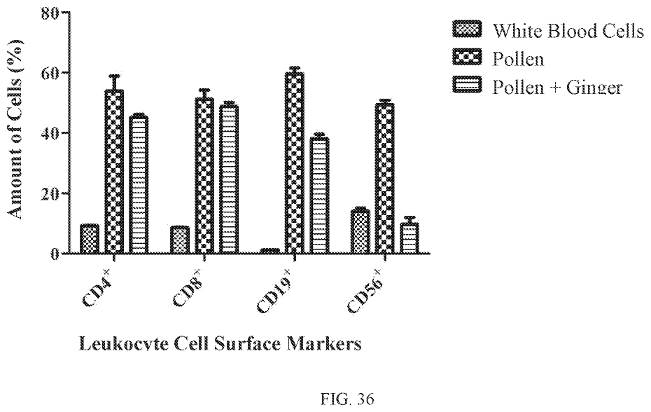

The cells grown in the medium are incubated with IL2, PHA, Mite Allergens and Pollen Allergens, thereby activating the immune system cells. The isolated plant exosomes are delivered to activated and unactivated blood cells. In order to demonstrate the effects of the plant exosomes on white blood cells, the percentages of the blood cells marked with CD4, CD8, CD19 and CD56 antibodies are measured by flow cytometry device. The effect of the Warty-Leaved Rhubarb exosomes on blood cells is shown in FIG. 7, the effect thereof on IL2 activated blood cells is shown in FIG. 8, the effect thereof on PHA activated blood cells is shown in FIG. 9, the effect thereof on Mite Allergen activated blood cells is shown in FIG. 10 and the effect thereof on Pollen Allergen activated blood cells is shown in FIG. 1. The effect of the Celery exosomes on blood cells is shown in FIG. 12, the effect thereof on IL2 activated blood cells is shown in FIG. 13, the effect thereof on PHA activated blood cells is shown in FIG. 14, the effect thereof on Mite Allergen activated blood cells is shown in FIG. 15 and the effect thereof on Pollen Allergen activated blood cells is shown in FIG. 16. The effect of the Pomegranate exosomes on blood cells is shown in FIG. 17, the effect thereof on IL2 activated blood cells is shown in FIG. 18, the effect thereof on PHA activated blood cells is shown in FIG. 19, the effect thereof on Mite Allergen activated blood cells is shown in FIG. 20 and the effect thereof on Pollen Allergen activated blood cells is shown in FIG. 21. The effect of the Leek exosomes on blood cells is shown in FIG. 22, the effect thereof on IL2 activated blood cells is shown in FIG. 23, the effect thereof on PHA activated blood cells is shown in FIG. 24, the effect thereof on Mite Allergen activated blood cells is shown in FIG. 25 and the effect thereof on Pollen Allergen activated blood cells is shown in FIG. 26. The effect of the Horseradish exosomes on blood cells is shown in FIG. 27, the effect thereof on IL2 activated blood cells is shown in FIG. 28, the effect thereof on PHA activated blood cells is shown in FIG. 29, the effect thereof on Mite Allergen activated blood cells is shown in FIG. and the effect thereof on Pollen Allergen activated blood cells is shown in FIG. 31. The effect of the Ginger exosomes on blood cells is shown in FIG. 32, the effect thereof on IL2 activated blood cells is shown in FIG. 33, the effect thereof on PHA activated blood cells is shown in FIG. 34, the effect thereof on Mite Allergen activated blood cells is shown in FIG. 35 and the effect thereof on Pollen Allergen activated blood cells is shown in FIG. 36.

Within the scope of the present invention, the advantages of using the effects of plant exosomes on the immune system as immune system enhancers, silencers and modulators against diseases can be listed as follows:
The exosomes are entirely of plant origin,
Activation is better than similar activators (IL2) and more successful than the substances obtained as similar chemicals,
It is possible to obtain large amounts thereof inexpensively,
The ratio of suppression of the activation of the blood cells is more successful than the substances obtained as similar chemicals,
They do not have the toxic effects to the liver and the other organs that the other drugs have as they are completely of plant origin.
They are a plant derived product which can be used in place of the stem cell treatment that is used for suppressing the immune system in tissue and organ transplantations,
Since they do not contain contaminants of plant, animal or chemical origin, side effects due to the said contaminants are not encountered,
The use of these exosomes replaces use of elements such as aluminum which is known to be harmful to body in case of overuse.

### REFERENCES

[5]. Ludwig, A. K. and B. Giebel (2012). “Exosomes: small vesicles participating in intercellular communication.” Int J Biochem Cell Biol 44(1): 11-15.
[6]. Iraci, N., T. Leonardi, F. Gessler, B. Vega and S. Pluchino (2016). “Focus on Extracellular Vesicles: Physiological Role and Signalling Properties of Extracellular Membrane Vesicles.” Int J Mol Sci 17(2): 171.
[7]. Stegmayr, B. and G. Ronquist (1982). “Promotive effect on human sperm progressive motility by prostasomes.” Urol Res 10(5): 253-257.
[8]. Gupta A, Punatar S, Mathew L, Kannan S, Khattry N. Cyclosporine Plus Methotrexate or Cyclosporine Plus Mycophenolate Mofetil as Graft Versus Host Disease Prophylaxis in Acute Leukemia Transplant: Comparison of Toxicity, Engraftment Kinetics and Transplant Outcome. Indian Journal of Hematology & Blood Transfusion 2016; 32(3):248-256. doi:10.1007/s12288-015-0577-3.

What is claimed is:

1. Plant derived exosomes, wherein the plant derived exosomes are configured for a prevention and a treatment of diseases by activating, suppressing and modulating cells of an immune system.

2. The plant derived exosomes according to claim 1, wherein the plant derived exosomes are obtained from at least one portion of a plant selected from the group consisting of an entire plant, a fruit, a leaf, a seed, a root, differentiated tissues like the plant's tissue culture medium, a stem cell, a waste material, a shell and phloem.

3. The plant derived exosomes according to claim 2, wherein the plant derived exosomes are obtained from a plant tissue culture in order to produce exosomes at a concentration up to 5 times higher than the plant derived exosomes obtained from similar plants and to maintain a content and properties of produced exosomes for a predetermined time, thereby preventing the plant derived exosomes from being affected by effects of the farm, harvesting, and transport.

4. The plant derived exosomes according to claim 3, wherein cells in the plant tissue culture are transfected to produce proteins, and the proteins enhance, suppress or modulate the immune system.

5. The plant derived exosomes according to claim 1, wherein the plant derived exosomes are isolated by an isolation method selected from the group consisting of an isolation by two phase liquid system, a graduated centrifuge, an ultrafiltration, chromatographic methods, a polymer based isolation and an isolation by microbeads.

6. The plant derived exosomes according to claim 1, wherein the plant derived exosomes are used in autoimmune diseases, and in cell, tissue, organ transplantations, in Graft Versus Host disease, and diseases affecting the immune system as immune system enhancers, immune system suppressors or immune system modulators, the diseases affecting the immune system comprise rheumatoid arthritis, juvenile rheumatoid arthritis, systemic lupus erythematosus, sclerosis, Sjogren's syndrome, Type 1 Diabetes, allergic asthma, Wegener granulomatosis, Multiple Sclerosis, Crohn's disease, psoriasis, Graves' disease, Celiac Disease, alopecia areata (pelade), central nervous system vasculitis, Hashimoto's thyroiditis, myasthenia gravis, Goodpasture's syndrome, autoimmune hemolytic anemia, Guillan-Barre syndrome, polyarteritis nodosa, idiopathic thrombocytic purpura, temporal arteritis, primary biliary cirrhosis, Addison's Disease, ankylosing spondylitis, Reiter's syndrome, Takayazu's arteritis, and vitiligo, wherein the immune system modulators perform functions of the immune system enhancers and the immune system suppressors.

7. The plant derived exosomes according to claim 1, wherein the plant-derived exosomes are administered orally, intranasally, intravenously, intramuscularly, intradermally, topically, intraperitoneally, and by an injection for administering an effective dose of selected plant exosomes to a patient to treat immune system-mediated diseases.

8. The plant derived exosomes according to claim 1, wherein the plant derived exosomes are configured for carrying immunomodulatory drugs.

9. The plant derived exosomes according to claim 1, wherein the plant derived exosomes are configured as adjuvants in vaccination applications.

10. The plant derived exosomes according to claim 1, wherein the plant derived exosomes are configured as a nutritional supplement for a purpose of modulating the immune system.
